(19) United States
(12) Patent Application Publication
(10) Pub. No.: US 2024/0115830 A1
(43) Pub. Date: Apr. 11, 2024

(54) Nose Covering with Scent

(71) Applicant: Tristan D. Murray, Louisville, KY (US)

(72) Inventor: Tristan D. Murray, Louisville, KY (US)

(21) Appl. No.: 18/376,731

(22) Filed: Oct. 4, 2023

Related U.S. Application Data
(60) Provisional application No. 63/414,109, filed on Oct. 7, 2022.

(51) Int. Cl. A61M 21/02

(52) U.S. Cl. CPC A61M 21/02; A61M 2021/0016

(57) ABSTRACT

A nose covering is disclosed. The nose covering includes a body having an exterior surface. The body is configurable to have a nose cavity for receiving a human nose. The body of the nose covering holds a scent substance, and the scent substance releases an aroma from the body to mask an unpleasant smell. The nose covering may also include a plurality of grooves in the exterior surface of the body for gripping the body.

### CROSS-REFERENCE TO RELATED APPLICATIONS

This application claims the benefit of U.S. Provisional Application No. 63/414,109, entitled “Nose Covering with Scent” and filed on Oct. 7, 2022. The complete disclosure of said patent application is hereby incorporated by reference.

### STATEMENT REGARDING FEDERALLY SPONSORED RESEARCH OR DEVELOPMENT

Not applicable

### BACKGROUND OF THE INVENTION

Changing an infant's diaper has long been an activity that has caused anxiety, even for the best of parents. There is no doubt the aroma produced by infant fecal matter, especially after the introduction of solid foods, is unpleasant at least and nauseating at its worst. It has been reported that nearly one-third of parents have faked napping in hopes that someone else would change a dirty diaper and that ten percent of parents have performed emergency meditation to mitigate diapering stress. Historic gender roles have designated this responsibility to the mother, but the modern father has become more involved in childcare, including diaper changes. According to a study by the U.S. Department of Health and Human Services, 90% of fathers who live with children under 5 years of age will change a diaper at least several times a week. It would be desirable to develop a nose covering to mask the smell of infant feces during diaper changes, which will further facilitate the intermingling of childcare responsibilities among parents with a shared distaste for the smell of infant feces.

### BRIEF SUMMARY OF THE INVENTION

This invention is directed to a scented nose covering. The nose covering includes a sphere, preferably made of foam, that fits snuggly over the nose. A scent, preferably an infused oil or injected gel, is applied to the nose covering. The aroma from the scent will diffuse from the nose covering during use and competitively bind odor receptors on the user's olfactory sensory neurons, thus masking the smell from the diaper. The distracting spherical shape and color of the nose covering, combined with an auditory contribution from the parent, also increases attentiveness and minimizes activity of the infant.

In one embodiment, the nose covering includes a body having an exterior surface. The body is configured to have a cavity that is sized to receive a human nose. The body of the nose covering holds a scent substance, and the scent substance releases an aroma from the body.

In another embodiment, the nose covering also includes a plurality of grooves in the exterior surface of the body for gripping the body.

These and other features, objects, and advantages of the present invention will become better understood from a consideration of the following detailed description of the preferred embodiments in conjunction with the drawings as described following:

### BRIEF DESCRIPTION OF THE DRAWINGS

FIG. 1 is a side view of the nose covering of the present invention.

FIG. 2 is a front view of the nose covering of the present invention.

FIG. 3 is a back view of the nose covering of the present invention with the nose covering in the closed position.

FIG. 4 is a back view of the nose covering of the present invention with the nose covering in the open position.

FIG. 5 is a top view of the nose covering of the present invention with the nose covering in the open position.

FIG. 6 is a perspective view of the nose covering of the present invention positioned on the nose of a user.

### DETAILED DESCRIPTION OF THE INVENTION

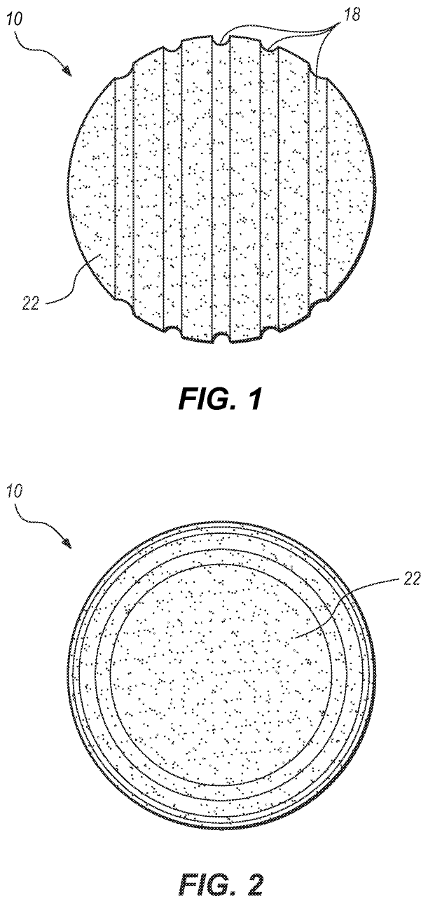

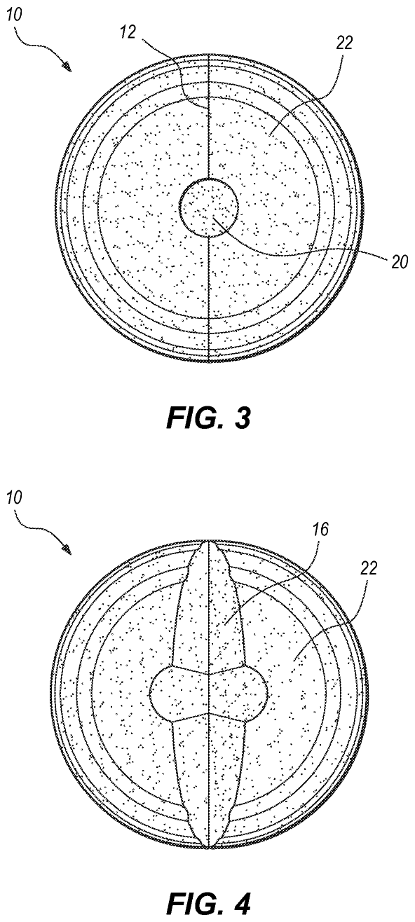

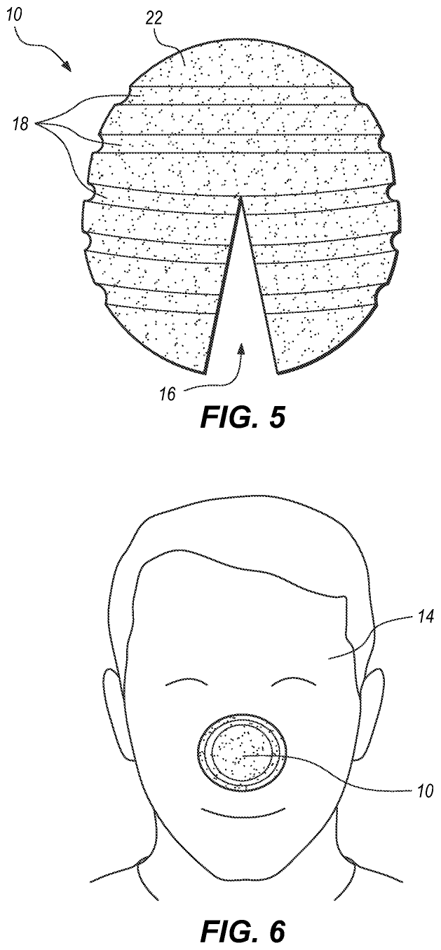

With reference to FIGS. 1-6, the preferred embodiments of the nose covering of the present invention may be described. The nose covering 10 is preferably made of spherically shaped body 22 that is preferably 2-3 inches in diameter. The body 22 is preferably made of foam. Alternatively, the body 22 may be made of another suitable nose covering material that is soft, resilient, and buoyant. The body 22 includes a slit 12 that allows the nose covering to be opened before being placed on the nose of a user 14. The slit 12 preferably is midline and extends from the top to the bottom on the back surface of the body 22. The back of the body 22 faces towards the user when the nose covering is worn. The slit 12 preferably extends approximately halfway through the body 22, as shown in FIG. 5. However, it should be understood that the slit 12 may extend more or less through the body for an optimal fit on noses with different shapes and sizes. When the body 22 on opposite sides of the slit 12 are pulled apart, a nose cavity 16 in the body 22 for receiving the nose of the user 14 is created, as shown in FIG. 4. In this regard, the slit 12 can be transitioned to a nose cavity 16 operable for receiving the nose of the user, as shown in FIGS. 3-4. The nose covering 10 may be referred to as being in the open position when a user is pulling apart the sides of the body, as shown in FIGS. 4-5. Alternatively, the nose covering 10 may be referred to as being in the closed position when the nose covering is not being used and the opposite sides of the body separated by the slit 12 are in contact, as shown in FIG. 3.

Grooves 18 are cut into the exterior surface of the body 22 of the nose covering 10, as shown in FIGS. 1 and 5. The grooves 18 are preferably circumferential, and they aid the user in gripping the body 22. The grooves 18 also aesthetically differentiate the nose covering from other nose coverings. In addition, these grooves 18 help camouflage imperfections in the body 22 made as a result of injecting scented gel from the exterior surface into the core of the body 22.

A channel 20 extends from the back of the body 22 into the middle of the body 22, as shown in FIG. 3. The channel 20 corresponds to the shape of a rod that extends into the body 22 during the manufacturing process. When the rod is removed, the channel is empty. However, the channel 20 also serves a functional purpose. In particular, the channel 20 aids the user in locating the slit 12 and allows the user to insert the tip of their finger to pull apart the sides of the body for transitioning the nose covering to the open position.

The nose covering 10 also includes a scent substance. The scent substance preferably is applied to the nose covering by infusing a scented oil into the body material (e.g., foam or similar material) prior to cutting the body or by injecting a scented gel (made from combining a semi-solid or wax with scented oil) into the body material. Examples of scents include, but are not limited to: cedar, coffee beans, and vanilla. The scented oil is infused, or the scented gel is injected, into the front end of the body 22 because the distal concentration of the scent allows balanced diffusion and better toleration by the user. However, it is permissible for the scented oil or scented gel to be infused or injected in one or multiple locations of the body 22.

The aroma that is released from the scent substance in the body 22 competitively binds odor receptors on olfactory sensory neurons of the user, thus masking the smell from the diaper. The distracting spherical shape and color of the nose covering 10, combined with an auditory contribution from the parent, also increases attentiveness and minimizes activity of the infant. The nose covering 10 preferably is stored in a capsule so that the scent does not fade as a result of being exposed to the environment. In one embodiment, the capsule is plastic and slightly larger than the nose covering.

### REFERENCES

1. PR Newswire. New survey finds parents go to great lengths to deal with dirty diapers—especially for stressful changes outside the nursery. News Provided By Munchkin; 6 Aug. 2019.
2. Fillo J, Simpson J A, Rholes W S, Kohn J L. Dads doing diapers: individual and relational outcomes associated with the division of childcare across the transition to parenthood. J Pers Soc Psychol 2015; 108(2):298-316.
3. Jones J, Mosher W D. Fathers' involvement with their children: United States, 2006-2010. U.S. Department of Health and Human Services: National Health Statistics Report 2013; Number 71.
4. National Institute on Deafness and Other Communication Disorders. Smell Disorders. NIH Pub. No. 09-3231; September 2013.
5. Singh V, Murphy N R, Balasubramanian V, Mainland J D. Competitive binding predicts nonlinear responses of olfactory receptors to complex mixtures. PNAS 2019; 116(19):9598-9603.
6. Tellinghuisen D J, Oakes L M. Distractibility in infancy: the effects of distractor characteristics and type of attention. Journal of Experimental Child Psychology 1997; 64(2):232-254.

The present invention has been described with reference to certain preferred and alternative embodiments that are intended to be exemplary only and not limiting to the full scope of the present invention.

We claim:

1. A nose covering comprising:
a body having an exterior surface, wherein said body is configurable to have a cavity operable for receiving a human nose; and
a scent substance, wherein said body holds said scent substance and wherein said scent substance releases an aroma from said body.

2. The nose covering of claim 1, wherein said body is made of a foam.

3. The nose covering of claim 1, wherein said scent substance comprises a scented oil.

4. The nose covering of claim 3, wherein said scented oil is infusible in said body.

5. The nose covering of claim 1, wherein said scent substance comprises a scented gel.

6. The nose covering of claim 5, wherein said scented gel is injectable in said body.

7. The nose covering of claim 1, wherein a back of said body comprises a slit.

8. The nose covering of claim 7, wherein said slit is reconfigurable to form said cavity.

9. The nose covering of claim 1, wherein said nose covering comprises an open position and a closed position.

10. The nose covering of claim 1, further comprising a channel extending partially into said body.

11. The nose covering of claim 10, wherein said cavity comprises said channel.

12. The nose covering of claim 1, wherein said nose covering further comprises a plurality of grooves in said exterior surface of said body for gripping said body.

13. A nose covering comprising:
a body having an exterior surface, wherein said body is configurable to have a cavity operable for receiving a human nose;
a plurality of grooves in said exterior surface of said body for gripping said body; and
a scent substance, wherein said body holds said scent substance and wherein said scent substance releases an aroma from said body.
